(19) United States
(12) Patent Application Publication
(10) Pub. No.: US 2024/0055013 A1
(43) Pub. Date: Feb. 15, 2024

(54) METHOD AND APPARATUS FOR DETERMINING A MEASURE OF SPEECH INTELLIGIBILITY

(71) Applicants: Katholieke Universiteit Leuven, Leuven (BE); Universidad de la Republica, Montevideo (UY)

(72) Inventors: Francisco Cervantes Constantino, Montevideo (UY); Tom Francart, Blanden (BE); Jonas Vanthornhout, Tildonk (BE)

(73) Assignees: Katholieke Universiteit Leuven, Leuven (BE); Universidad de la Republica, Montevideo (UY)

(21) Appl. No.: 17/766,172

(22) PCT Filed: Oct. 2, 2020
(86) PCT No.: PCT/EP2020/077699

§ 371 Date: Apr. 1, 2022

(30) Foreign Application Priority Data
Oct. 4, 2019 (GB) 1914360.1
Oct. 25, 2019 (EP) 19205337.9

(51) Int. Cl. G10L 21/0364; G06F 3/01

(52) U.S. Cl. CPC G10L 21/0364; G06F 3/011

(57) ABSTRACT

A method of estimating speech intelligibility is disclosed. The method comprises the steps of providing at least a first time-dependent signal derived from a first auditory stimulus and a corresponding first measured EEG response; comparing at least part of the first signal with at least part of the first measured EEG response in order to determine a signal-response latency difference; comparing the signal-response latency difference to a reference value; and deriving a measure of speech intelligibility based on the comparison of the signal-response latency difference and the reference value.

### FIELD OF THE INVENTION

The present invention relates to methods and apparatus for determining an objective measure of speech intelligibility based on an EEG response to an auditory stimulus.

### BACKGROUND OF THE INVENTION

Hearing tests can involve playing a sound such as a speech fragment to a test subject and determining their response. One simple way of doing this is to simply ask the test subject what they heard, and determining the intelligibility of the speech based on this response. This is termed a behavioural measure of speech intelligibility. However, this is impractical or impossible in some situations, for example where the test subject is a young child or a disabled person. Furthermore, such methods are by nature subjective.

By measuring the response of the test subject's neural system to the sound using an electroencephalogram (EEG), an objective measure of speech intelligibility can be obtained.

“Adaptive Temporal Encoding Leads to a Background-Insensitive Cortical Representation of Speech”, Ding et al., Journal of Neuroscience 27 Mar. 2013, 33 (13) 5728-5735, describes that speech intelligibility is correlated with the SNR of the speech sample.

“EEG can predict speech intelligibility”, lotzov et al., Journal of Neural Engineering, Volume 16, Number 3 (2019), describes correlating the amplitude of noisy speech with the corresponding brain responses and predicting intelligibility from the EEG.

In “Speech Intelligibility Predicted from Neural Entrainment of the Speech Envelope”, Journal of the Association for Research in Otolaryngology, 19 , 181-191 (2018), a backward model is described wherein by decoding the auditory stimulus from the neural activity as measured using EEG, a comparison between the decoded and the actual speech signals can yield a tracking performance measure. Using the speech envelope, the decoding accuracy of the backward model was used to predict behaviourally measured individual speech reception thresholds (SRT). The SRT is a clinically used measure of speech understanding and is the stimulus signal-to-noise ratio (SNR) at which the subject understands 50% of the words. Decoding accuracy performance was measured as the correlation between the actual and the decoded speech envelope. This accuracy increased with improving SNR.

A practical issue with this approach is the relatively long duration from the decoder to performance estimation. 15 minutes of data is needed to train a decoder before any decoding accuracy can be calculated. It is also limited in interpretation as it measures how the EEG signal encodes the stimulus, rather than measuring the operation of the auditory system itself. Conceptually, backward decoding methods assume EEG-stimulus transfer functions to be relatively stationary across conditions, such as different noise levels. One decoder was used, trained in one condition to estimate decoding accuracies in different conditions.

WO 2018/160992 A1 describes a method for determining the cognitive function of a subject. The method includes receiving, by a processor, a measurement of a neural response of a subject to one or more naturalistic sensory stimuli.

There is still a need for a method of predicting speech intelligibility which is reproducible across listeners and correlates well with behavioural measures of speech intelligibility.

### SUMMARY OF THE INVENTION

According to a first aspect of the present invention, there is provided a method of estimating speech intelligibility comprising providing at least a first time-dependent signal derived from a first auditory stimulus, preferably wherein the first auditory stimulus has a first noise rating, and a corresponding first measured EEG response; comparing at least part of the first signal with at least part of the first measured EEG response in order to determine a signal-response latency difference; comparing the signal-response latency difference to a reference value; and deriving a measure of speech intelligibility based on the comparison of the signal-response latency difference and the reference value. The reference value is preferably a second signal-response latency difference. The second signal-response latency difference is preferably obtained by providing a second signal derived from a second auditory stimulus, wherein the second auditory stimulus preferably has a second noise rating which is different to the first noise rating, and a corresponding second measured EEG response; and, comparing at least part of the second signal with at least part of the second measured EEG response in order to determine a second signal-response latency difference

Therefore, the method according to the invention preferably comprises:

providing at least a first time-dependent signal derived from a first auditory stimulus wherein the first auditory stimulus has a first noise rating and a corresponding first measured EEG response;

comparing at least part of the first signal with at least part of the first measured EEG response in order to determine a first signal-response latency difference; providing a second signal derived from a second auditory stimulus, wherein the second auditory stimulus has a second noise rating which is different to the first noise rating, and a corresponding second measured EEG response; comparing at least part of the second signal with at least part of the second measured EEG response in order to determine a second signal-response latency difference;

comparing the first signal-response latency difference to the second signal-response latency difference; and

deriving a measure of speech intelligibility based on the comparison of the first signal-response latency difference and the second signal-response latency difference.

It is an advantage of embodiments of the present invention that an objective measure of speech intelligibility can be determined without requiring cooperation or input of the subject. Method according to embodiments of the present invention can be used with subjects who are unable to communicate, such as young children or those with a disability.

It is an advantage of embodiments of the present invention that the discovered relation between the latency and behaviourally measured speech intelligibility can be used to predict speech intelligibility simply by performing processing as described herein on measured EEG responses and auditory stimuli. This enables a fast and inexpensive determination of speech intelligibility which can be evaluated without requiring the presence of an audiologist.

It is an advantage of embodiments of the present invention that, by not relying on measuring a subjective response to the stimulus, bias in the response of the subject can be avoided. For example, a subject may not feel confident in their hearing and may state that they did not understand a statement where in fact some understanding is present. A subject may wish to simulate hearing loss where in fact none exists.

It is an advantage of embodiments of the present invention that no standard values of the population are needed to perform the method. The method can be performed on an isolated subject.

It is an advantage of embodiments of the present invention that by using the change in latencies, the present invention is less susceptible to individual differences.

The method may comprise the step of comparing at least part of the first signal and at least part of the EEG response comprises determining a temporal response function for predicting at least part of the EEG response based on the first signal.

The step of comparing at least part of the first signal and at least part of the EEG response may comprise applying a plurality of test latency differences to the first signal or to the EEG response with respect to the first signal, and determining a quantitative measure of the similarity of at least part of the first signal and at least part of the EEG response for each applied test latency difference, and wherein the signal-response latency difference is determined as the latency difference at which the quantitative similarity measure is greatest.

The first auditory stimulus may have a first noise rating and the method may further comprise providing a second signal derived from a second auditory stimulus, wherein the second auditory stimulus has a second noise rating value which is different to the first noise rating, and a corresponding second measured EEG response; comparing a feature of the second signal and a corresponding feature of the second EEG response in order to determine a feature latency difference between the features; wherein the reference value is the feature latency difference determined for the first signal and first corresponding measured EEG response.

The first auditory stimulus may have a first noise parameter value and the method may further comprise providing a second time-dependent signal based on a second auditory stimulus having a second noise rating which is different to the first noise rating and a corresponding second measured EEG response; comparing at least part of the second time-dependent signal and at least part of the second EEG response, wherein the comparison comprises determining a temporal response function for predicting at least part of the second EEG response based on the second signal; and performing a cross-correlation of the first TRF (temporal response function) and the second TRF (temporal response function) in order to determine the signal-response latency difference as a relative signal-response latency difference. Preferably, the relative signal-response latency difference is the latency of the maximum or minimum of the cross-correlation.

The use of TRFs has the advantage over the use of cross-correlation in that TRFs provide a more detail on the signal-response latency.

The second auditory stimulus may be noise-free.

The method may comprise comprising providing a third time-dependent signal derived from a third auditory stimulus, wherein the third auditory stimulus has a third noise rating which is different to the first noise rating and the second noise rating, and a corresponding third measured EEG response; comparing at least part of the third signal and at least part of the third EEG response in order to determine a third signal-response latency difference; comparing the third signal-response latency difference signal to a reference value; and deriving a measure of speech intelligibility based on the comparison of the third signal-response latency difference and the reference value. Preferably the third signal-response latency difference is compared to the first and/or second signal-response latency difference.

The noise rating may be a signal-to-noise ratio.

The reference value for the first and second signals may be the latency difference associated with the third auditory stimulus.

The reference value may be an average latency difference of a sample dataset of auditory stimuli and corresponding EEG responses. The step of comparing the signal-response latency difference to a reference value may comprise supplying the signal-response latency difference and the reference value as inputs to a comparison function which produces a single output so as to obtain at least two sets of a comparison function output and a corresponding noise rating for the respective stimulus, and wherein determining a measure of speech intelligibility comprises fitting a function to the comparison function output-noise rating data and determining the speech intelligibility based on a parameter of the fitted function.

The step of comparing the signal-response latency differences may comprise supplying the signal-response latency differences as inputs to a comparison function which produces a single output. The term “comparison function” may also be referred to as “transformation function”.

The method may comprise the step of obtaining at least at least two sets, for example a set for the first auditory stimulus and a set for the second auditory stimulus, each set comprising a comparison function output and a corresponding noise rating for the respective stimulus.

The step of determining a measure of speech intelligibility may comprise fitting a function to the comparison function output-noise rating data and determining the speech intelligibility based on a parameter of the fitted function

The comparison function may compute the signal-response latency difference divided by the reference value or the difference between the reference value and the signal-response latency difference.

The comparison function may compute the first signal-response latency difference divided by the first signal-response latency difference or vice versa, or the difference between the signal-response latency differences.

The fitted function may be a linear or an exponential or a sigmoid function.

The signal may be the envelope of the stimulus or the derivative of the envelope of the stimulus.

According to a second aspect of the present invention there is provided a computer program comprising instructions which, when the program is executed by a computer, cause the computer to carry out the method of the first aspect.

According to a third aspect of the present invention there is provided a computer-readable medium comprising instructions which, when executed by a computer, cause the computer to carry out a method according to the first aspect. According to a fourth aspect of the present invention there is provided an apparatus comprising a control module, wherein the control module comprises a processor for carrying out a method according to the first aspect. The control module may comprise one or more inputs for receiving the auditory stimulus and EEG response.

Particular and preferred aspects of the invention are set out in the accompanying independent and dependent claims. Features from the dependent claims may be combined with features of the independent claims and with features of other dependent claims as appropriate and not merely as explicitly set out in the claims.

### BRIEF DESCRIPTION OF THE DRAWINGS

Further features of the present invention will become apparent from the examples and figures, wherein:

FIG. 1a is a schematic diagram of a setup for measuring an EEG response to an auditory stimulus and performing processing of the auditory stimulus and EEG response;

FIG. 1b shows a plot of an auditory stimulus and an EEG response measured in response to the stimulus;

FIG. 2 is a flow chart of a method according to embodiments of the present invention;

FIG. 3a shows a subset of TRFs from a representative subject depicting gradual delaying, as signal-to-noise lowers, in at least two peaks: an early (from <100 ms for clean speech), negative-polarity TRF-early peak, as well as a positive, late (>180 ms) prominent TRF-late peak;

FIG. 3b shows TRFs trained after envelope representations of speech, which show noise-induced change, including on the latency, to the TRF-50 and TRF-100 peaks in a representative subject;

FIG. 4a shows an exponential model fit to the latency versus SNR for the TRF-early (left panel) or the TRF-late components (right panel);

FIG. 4b shows an exponential model fit in the envelope case for the TRF-100 peak. Given the observed lack of TRF-50 in the Story and 100 dB SNR conditions, noise-induced delays were modeled with a linear regression in this noise level range;

FIG. 5a shows TRF-early curves for 12 subjects. While they may show different initial latencies, delay rates by noise appear similar across these participants;

FIG. 5b shows TRF-late curves for 28 subjects. This peak typically began after 150 ms, and showed similar latency delay rates by noise across subjects;

FIG. 6a shows the delay rates for both TRF-early and TRF-late peaks;

FIG. 6b shows the relationship between a behavioural measure of speech intelligibility (BMSI) and an objective measure of speech intelligibility (OMSI) as calculated using methods described herein. The intersect of individual TRF-late trendlines, computed between the delay ratio of each noise level referenced to noise-free limit levels (expressed in dB) and SNR, serves as basis for an objective measure of speech intelligibility. Its inverse was found to correlate with the speech reception threshold reciprocal, which was used as a behavioural measure of speech intelligibility. Each symbol is the result from a subject.

### DETAILED DESCRIPTION OF CERTAIN EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. Where an indefinite or definite article is used when referring to a singular noun e.g. “a” or “an”, “the”, this includes a plural of that noun unless something else is specifically stated. The term “comprising”, used in the description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein. In the drawings, like reference numerals indicate like features; and, a reference numeral appearing in more than one figure refers to the same element.

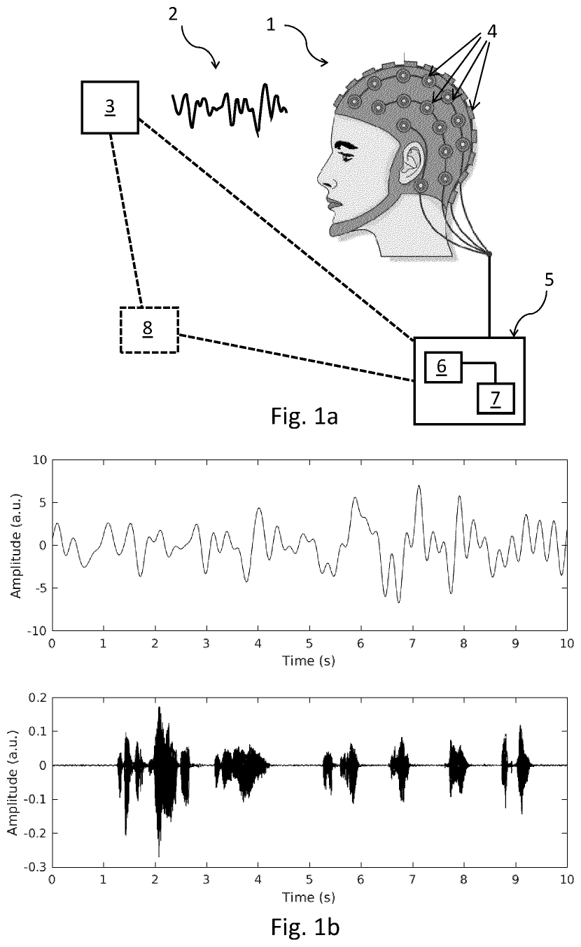

Referring to FIG. 1a, in a setup for a hearing test, a test subject 1 listens to an auditory stimulus 2 from a sound source 3. The sound source can be a speaker at a distance from the subject 1, or a set of headphones or earphones worn by the subject. EEG probes 4 are attached to the subject's head for measuring the neural response provoked by the auditory stimulus 2. The subject may be asked to describe the auditory stimulus after listening to it, for example to specify the words spoken if the stimulus is a speech sample. The EEG probes 4 provide signals to a control module 5 which is configured to receive and process signals from the EEG probes 4. The control module 5 may additionally be configured to control the sound source 3, for example to trigger playback of the auditory stimulus. The control module comprises a memory 6 for storing the auditory stimulus and/or a signal derived from the auditory stimulus and received EEG signals, and a processor 7 for processing the auditory stimulus and/or signal derived from the auditory stimulus, and received EEG signals. The minimum number of EEG probes required is two: an active probe and a reference probe. Herein, where reference is made to an EEG response, it is to be understood as meaning the measurement of at least one EEG channel, where measuring each EEG channel requires a respective active probe and corresponding reference probe. The EEG response may be processed before carrying out a method as described herein, for example by filtering and/or normalising and/or re-referencing the EEG response. Herein, where at least part of the EEG response is referred to, it is understood that this can refer to a processed subset of the EEG response e.g. a filtered EEG response, a subset in time of the EEG response, a normalised EEG response. The first auditory stimulus may be provided as an input to the control module 5 from an optional separate control module 8.

Referring to FIG. 1b, an example auditory stimulus and corresponding EEG response are shown. The neural response to a particular feature of an auditory stimulus is offset in time with respect to the position in time of the feature of the auditory stimulus. The position in time of a feature, relative to a time t=0 being the time when the auditory stimulus begins, is referred to as the latency. The latency of a time-dependent signal derived from the auditory stimulus and the latency the EEG response, caused by the auditory stimulus, are different as the auditory system of the test subject does not instantaneously process the auditory stimulus.

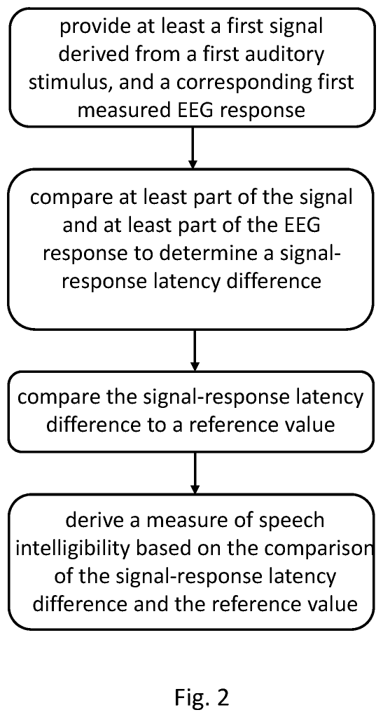

Referring to FIG. 2, in a method according to embodiments of the present invention, the following steps are carried out.

In step S1, a first time-dependent signal derived from a first auditory stimulus is provided. For example, the first signal may be stored in the memory 6 of the control module 5. The first auditory stimulus may be stored in the memory 6 of the control module 5 and, in a pre-processing step, may be loaded into the processor 7 for deriving the first signal. The first auditory stimulus may be provided as an input to the control module 5 from an optional separate control module 8 for providing signals to the sound source 3 via a wired or wireless connection and, in a pre-processing step, the first signal may be derived from the first auditory stimulus by the processor 7. Additionally, a corresponding first measured EEG response is provided. The corresponding first measured EEG response comprises measurements from the EEG probes 4 as measured at least during the period during which the auditory stimulus is played to the test subject, and may also include measurements from the EEG probes 4 as measured just before the first auditory stimulus is played to the test subject and/or for a period of time after the end of the auditory stimulus. The control module 5 need not be physically present during the hearing test and may receive the auditory stimulus and/or first signal, and corresponding first measured EEG response remotely through a wired or wireless connection.

The signal derived from the auditory stimulus is time-dependent, meaning that it exhibits variation in time. Examples of suitable signals are time-frequency and time-amplitude signals. For example, the signal may be the temporal envelope of the auditory stimulus, which can be derived from the auditory stimulus by a rectification step followed by application of a low-pass filter. The signal may be the spectrogram of the auditory stimulus, i.e. the stacked envelopes of multiple frequency bands. The signal may be related to phonetic features, for example may be a representation of the point in time at which a certain phoneme is present. For example, the signal may have a non-zero amplitude at points in time where a phoneme is present in the auditory stimulus and a zero amplitude at all other times. Similarly the signal may be related to word frequencies, semantic features, and/or syntactic features.

Without wishing to be bound by theory, the time-dependent signal can be thought of as a time-domain representation of the auditory stimulus corresponding to the representation of the auditory stimulus at a certain stage of processing in the brain. In embodiments of the present invention wherein the first auditory stimulus is provided and a pre-processing step is performed to derive the first signal, the pre-processing step may comprise a rectification step and/or a filtering step. The pre-processing step may comprise performing a Hilbert transform of the stimulus and taking the absolute value of the transformed stimulus. The pre-processing step may comprise using a filterbank approach, such as a gammatone filterbank, and calculating the envelope per frequency band. The pre-processing step may comprise applying an auditory periphery model. The pre-processing step may comprise applying logarithm, power law or square root compression or no compression. The pre-processing step may comprise extracting the envelope by performing a method as described in Biesmans et al., Auditory-Inspired Speech Envelope Extraction Methods for Improved EEG-Based Auditory Attention Detection in a Cocktail Party Scenario. IEEE Trans Neural Syst Rehabil Eng. 2017, 25(5):402-412.

One or more further pre-processing steps may be performed on the auditory stimulus and/or the measured EEG response. For example, as will be described in more detail hereinafter, the auditory stimulus may initially be in a sound file format, such as mp3 or wav, suitable for playback through a sound source, and the pre-processing step may include processing the sound file to extract a vector of amplitudes and times. In some embodiments, the signal is an envelope onset representation of the auditory stimulus and the pre-processing step comprises obtaining the envelope onset representation by differentiating the acoustic envelope and then applying a half-wave rectification.

The signal may be the spectrogram of the auditory stimulus. In this case, the pre-processing step comprises spitting the auditory stimulus into multiple frequency bands and calculating the envelope per frequency-band. The envelope for each frequency band can be extracted as described hereinbefore.

The signal may be based on a word embedding such as GloVe, Bert, Word2vec, fasttext and Elmo. These are databases that contain a multi-dimensional vector for each (part of) a word. Each word, or part of word, of the auditory stimulus can be replaced by the corresponding multi-dimensional vector. This can be done for the complete duration of the word, or only for the beginning/end of the word. As these database have a high dimensionality, a dimensionality reduction can first be performed by carrying out a principal component analysis and carrying out the replacement as described above on the lower dimension data resulting from the principal component analysis.

The signal may be based on a semantic dissimilarity. The signal can be obtained by correlating the multi-dimensional vector of a specific word from one of the previously mentioned databases with the average multi-dimensional vector of n words preceding the selected word. This value is subtracted from 1 to obtain a number between 0 and 2. The signal is equal to this value for the complete duration of the word or alternatively only at the beginning/end of this word.

The present invention is not limited to using the amplitude envelope, but can also use phonetic information, semantic dissimilarity, syntactic depth etc. The advantage of this is that it can more easily pinpoint disorders. For example, if by using the amplitude envelope it is found that the subject has normal speech intelligibility, but by using semantic dissimilarity it is found that the subject has bad speech intelligibility, it may be concluded that the subject has normal hearing but has a problem with processing speech signals.

The signal may be based on the onset of one or more words; the signal then comprises a pulse at the start of each of the one or more words, the remainder of the signal being zero.

The signal may be based on a syntactic depth feature as follows. The auditory stimulus is converted into a syntax tree and then the value of the depth of each word in this tree is placed in the signal for the complete duration of the word or just the beginning/end of this word.

The EEG response may be spectrally and/or spatially filtered in order to improve the signal to noise ratio of the EEG response.

In step S2, at least part of the signal and part of the first measured EEG response are compared in order to determine a signal-response latency difference between the signal and the first measured EEG response. As will be described in more detail hereinafter, the comparison may involve applying a plurality of test latency differences to the EEG response with respect to the signal and determining a quantitative measure of the similarity of at least part of the signal and at least part of the EEG response for each applied test latency difference, and determining the signal-response latency difference as the latency difference at which the quantitative similarity measure is greatest. The comparison may take the form of a cross-correlation of the signal and the measured EEG response. The comparison may take the form of determining a temporal response function for predicting the EEG response based on the signal.

Each feature will have a weight for each latency. We take the latency of the highest/lowest (depending on if you want negative or positive peaks) value in a certain window. The window is defined by which peak you want: early or late.

In embodiments wherein step S2 comprises determining a temporal response function, the following steps are carried out. Each EEG response, that is, the EEG response for each channel, is modelled as a linear combination of time-dependent signals, for example the envelope of the auditory stimulus, where each instance of the time-dependent signal is delayed by a different amount. The linear combination may also include an error correction term. The temporal response function, or TRF, is then a vector of weighting factors, or amplitudes, for each corresponding latency value. The TRF therefore varies as a function of latency of the envelope. The signal-response latency difference is determined as the latency corresponding to the maximum, or the minimum, of the weighting factors. The TRF may have positive or negative peaks and the choice of which peak to use for determining the signal-response latency difference depends on whether an early (approx <100 ms) peak is required, which is normally negative, or a late (approx >100, <200 ms) peak, which is normally positive.

In some embodiments, a single EEG channel response can be used to determine a TRF which is then used in the further analysis. In some embodiments, multiple EEG channel responses and/or multiple TRFs are used. For example, spatial filtering can be used to obtain one “combined” EEG channel, which is a linear combination of all the channels, and this is used to calculate one TRF. In some embodiments, one channel can be selected for the calculation of a TRF. In some embodiments, the EEG responses from multiple channels can be averaged and then used to calculate a TRF. In some embodiments, multiple EEG channels can be used for determining a corresponding TRF for each channel and multiple corresponding signal-response latency differences can be determined, and then averaged to give one signal-response latency difference.

In some embodiments, a reference TRF may be calculated in the same manner based on a reference auditory stimulus and corresponding response, and the reference TRF can be cross-correlated with the first signal TRF. The signal-response latency difference may then be the latency corresponding to the maximum (or the minimum) of the cross-correlation.

In step S3, the signal-response latency difference determined in step S2 is compared to a reference value.

The reference value may be a signal-response latency difference of a population with one or more characteristics in common with the test subject, for the same auditory stimulus. For example, the reference value may be the signal-response latency difference as determined for a population of the same age and sex as the test subject. Other characteristics that can match include ear preference, hand preference (left handed/right handed), IQ, reading ability, disorder type (Alzheimer, dyslexia, aphasia) if any is present.

The signal-response latency differences for the population may be averaged to obtain the reference value. To reduce the effect of outliers, a trimmed mean can be used instead, being the spectrum between the average (no trimming) and the median (full trimming).

In some embodiments the reference value may be a signal-response latency difference for a single person with one or more characteristics in common with the test subject, for the same auditory stimulus.

The reference value may be a signal-response latency difference determined for a second time-dependent signal derived from a second auditory stimulus, for example using a method of deriving a signal as described hereinbefore, and corresponding second EEG response. The second auditory stimulus is preferably noise-free but may in other embodiments have a non-zero signal to noise ratio. Put more generally, in embodiments wherein the reference value is a signal-response latency difference determined for a second signal and corresponding second EEG response, the first auditory stimulus and the second auditory stimulus have a different noise rating. The noise rating may be, for example, the signal to noise ratio (SNR), an amount of reverberation measured in terms of reverberation time; an amount of filtering of the auditory stimulus e.g. by removing or attenuating parts of the spectrum measured in terms of an amount of clipping measured in terms of a percentage of clipped samples; an amount of distortion introduced by hearing aid noise suppression measured in terms of a numerical value representing the amount of distortion, for example using a perceptual evaluation of speech quality (PESO) model; an amount of vocoding measured in terms of the number of bands. The noise rating in each case is defined with respect to the auditory stimulus in question when consisting of clean speech, that is, without noise or reverberation or other intelligibility-reducing perturbations. For example, the signal to noise ratio is defined as the power ratio between an auditory stimulus and the clean speech version of the auditory stimulus in question.

The noise rating may be a classification of the stimulus into a predefined category. For example, a stimulus may be labelled as “very distorted” “somewhat distorted” and “clean”, with numerical labels 3, 2 and 1 respectively assigned to each class. The labelling may be obtained by playing the stimulus to multiple listeners and asking them to rate each stimulus and averaging the results. The actual numerical labels can be chosen arbitrarily provided that there is a monotonic increase in the number with increasing distortion.

The comparison of the feature latency difference with the reference value comprises supplying the feature latency difference and the reference value as inputs to a comparison function. The comparison function operates on the feature latency difference and the reference value to generate a single output value. The operation of the comparison function may be, for example, division of the feature latency difference by the reference value, subtraction of the feature latency difference from the reference value, taking the logarithm of the division or subtraction.

In step S4, a measure of speech intelligibility is determined based on the comparison of the feature latency difference and the reference value as performed in step S3.

In some embodiments, the measure of speech intelligibility is determined based on an already known speech intelligibility-latency relation, for example a previously determined relation based on a set of behavioural measurements of SI and corresponding latencies, which may be calculated as described hereinbefore. In this case, the signal-response latency difference is compared to the latency values of the known relation to find a match, and the measure of speech intelligibility is determined as the speech intelligibility associated with the matching latency. In some embodiments, a function can be fitted to the already known SI-latency values and the signal-response latency can be input into the function to determine the speech intelligibility. A normalisation can be applied to the already known SI-latency values to account for latency differences between subjects.

In embodiments wherein more than one pair of auditory stimulus-EEG response pairs are provided, step S3 results in a set of comparison function output and noise parameter value data pairs, where each comparison function output is associated with a corresponding noise parameter value, the noise parameter value being that of the auditory stimulus used to calculate the feature latency difference provided as input to the comparison function. For example, in embodiments wherein a first, second, and third pair of auditory stimulus and corresponding EEG response are provided and wherein one of the auditory stimuli has a noise parameter value of zero corresponding to clean speech and is therefore used as a reference, two comparison function outputs each associated with a respective noise parameter value are generated in step S3. Step S4 may then comprise fitting a function to the comparison function output-noise parameter value data, for example a linear, exponential, or sigmoid function. The measure of speech intelligibility may then be determined in dependence upon a parameter of the fitted function. For example, if the fitted function is an exponential function, the measure of speech intelligibility may be determined based on an amplitude parameter of the exponential function. If the fitted function is a sigmoid function, the measure of speech intelligibility may be determined based on the midpoint of the sigmoid function. If the fitted function is a linear function, the intercept and/or the gradient can be used to determine the measure of speech intelligibility.

The measure of speech intelligibility may be the speech reception threshold (SRT), the percentage or number of words repeated correctly, an intelligibility rating, the percentage or number of sentences repeated correctly, the percentage or number of keywords repeated correctly.

The measure of speech intelligibility may be a relative measure. For example, in some embodiments, a first signal derived from a first auditory stimulus and corresponding EEG response are provided (step S1). A temporal response function is determined for predicting the first EEG response based on the first signal (step S2). A feature latency difference is determined by cross-correlating the TRF with a TRF of a population. The feature latency difference is the time at which the cross-correlation of the TRFs is at a maximum (step S3). This feature latency difference can then be used in the subsequent steps of the method according to embodiments of the present invention.

The present invention also comprises a computer-implemented method as described herein, and embodiments thereof. The present invention also comprises a method as described herein, and embodiments thereof, carried out by a computer.

The present invention also comprises a computer program (product), the computer program (product) comprising instructions which, when the program is executed by the computer, cause the computer to carry out a method as described herein, and embodiments thereof.

The present invention also comprises a computer comprising a computer program or a computer-readable medium, the computer program or computer-readable medium comprising instructions which, when the program is executed by the computer, cause the computer to carry out a method as described herein, and embodiments thereof.

The present invention also comprises an apparatus (or system or device) configured to carry out a method as described herein, and embodiments thereof, the apparatus (or system or device) comprising at least a control module for receiving and processing an auditory stimulus and an EEG response. Optionally, the apparatus also comprises EEG probes.

### EXAMPLE

### Subjects

28 subjects (19 female; mean age 23.4±2 years) participated voluntarily across two study protocols, 18 of them (14 female; mean age 22.7±1.8 years) in protocol 1, and 10 (5 female; mean age 24.6±2 years) in protocol 2. All were native Dutch (Flemish) speakers, and reported normal hearing as verified by pure tone audiometry (pure tone thresholds <25 dB HL in the 125-8000 Hz range) using a MADSEN Orbiter 80 922 audiometer (Madsen Ltd., Budapest, Hungary).

### Setup and Recordings

Experiments were conducted in an electromagnetically shielded and soundproofed room. Stimuli were presented using the APEX 3.1 software platform developed at the ExpORL research group (KU Leuven), an RME Multiface II sound card (Audio AG, Haimhausen, Germany), and Etymotic ER-3A insert phones (Etymotic Research Inc., Elk Grove Village, United States) which were electromagnetically shielded with a CFL2T screening can enclosure (Perancea Ltd., Perivale, United Kingdom). Sound was presented at 60 dBA and the setup was calibrated in a 2 cm³ BK 4152 coupler (Brüel & Kjær, Naerum, Denmark) using stationary speech-weighted noise corresponding to the speech material.

EEG signals were recorded with an ActiveTwo 64 channel system (BioSemi, Amsterdam, The Netherlands) with an extended 10/20 layout at 8192 Hz digitization rate. Experimental sessions lasted approximately 2 hours in total.

### Stimulus Materials

In both behavioral and EEG experiments, stimuli consisted of sentences from the Flemish Matrix speech material (Lots, H., Jansen, S., Dreschler, W., & Wouter, J. Development and Normative Data for the Flemish/Dutch Matrix Test, Technical Report, 2015), a standardized corpus of sentences validated for speech intelligibility tests. It is divided in lists of 20 sentences, each following a fixed name-verb-numeral adjective-object structure, with structure elements drawn from a pool of 10 alternatives each. These sentences are grammatically trivial but completely unpredictable, making them very useful in multiple repetitions. Sentences were produced by a female speaker and presented diotically.

For the EEG recordings, trials were created by concatenating two lists of Flemish Matrix sentences (40 sentences), separated by silent gaps uniformly distributed between 0.8 and 1.2 s long. Trials lasted approximately 120 s and were repeated 3-4 times each. Varying intelligibility conditions were created with additive noise set at ten signal-to-noise ratio (SNR) levels. For protocol 1 these 105 were −12.5, −9.5, −6.5, −3.5, −0.5, +2.5 and +100 dB (7 levels); and for protocol 2: −9.5, −7.6, −5.5, −1 and +100 dB (5 levels). Noise was stationary and filtered to the same average spectrum as speech. Presentation order of conditions was randomized across subjects. To keep subjects attentive, questions about the stimuli were asked after each condition.

In addition, subjects listened to a 14.5 minute-long Flemish children's story, Milan, narrated by Stijn Vranken, presented without noise. This condition is referred to as “Story”, for which subjects were not required to perform any task.

In the behavioral experiment the SRT was determined by collecting the percentage of correctly repeated words at different SNRs around the SRT, and fitting a sigmoid to the resulting percentage correct as function of SNR. The function was according to equation 1:

$$S ( SNR ) = {( 1 + {e}^{\frac{- SNR - α}{β}} )}^{- 1} \qquad ( 1 )$$

with S(SNR) being the word score at that SNR. The value of the SRT is equal to the α-parameter corresponding to the curve midpoint.

### Signal Preprocessing

The recordings, or auditory stimuli, and the corresponding measured EEG responses were provided to a computer (step S1). Data analysis was implemented in MATLAB 2016b (The Mathworks, Natick, USA). The EEG signal was downsampled offline to 512 Hz using the downsample function of MATLAB in order to speed up the following processing. Furthermore, the EEG signal was re-referenced to the scalp average signal for further data analysis.

### Spectral Filtering

Each EEG signal was filtered between 1 and 30 Hz with a fourth-order FIR Hamming window filter and corrected for group delay. EEG data was decomposed into independent components using the FastICA algorithm. Two independent components were automatically selected for their maximal proportion of broadband power in the 10-30 Hz region and projected out of the raw data.

### Spatial Filtering

In order to improve the SNR of the EEG while reducing dimensionality of the data, a spatial filter was constructed emphasizing signals that reflect reproducible activity across subjects. The data-driven joint decorrelation was trained on all 28 listeners' recordings during the “Story” condition, each organized in an 870 s epoch downsampled to 32 Hz. The outcome of this process is that a single linear spatial combination is found approximating the grand-average EEG signal. After filtering individual subject's EEG, the EEG component with the highest evoked/induced activity ratio was used for all subsequent analysis.

### Stimulus Representations

The acoustic envelope of the auditory, or speech, stimulus was extracted using a 28-channel gammatone filterbank spaced by 1 equivalent rectangular bandwidth and centre frequencies ranging from 50 Hz until 5000 Hz. Sub-band absolute values were raised to the power of 0.6, with resulting signals averaged to obtain the overall envelope, which was downsampled to the sample rate of the EEG signal.

An envelope onset representation of the auditory stimulus was obtained by differentiating the envelope, followed by half-wave rectification, resulting in a signal which is proportional to the energy change in low-frequency speech modulations. All subsequent analyses were conducted using the onset envelope representation and, where indicated, the regular envelope.

### Temporal Response Function Estimation

To address the correspondence between the slow dynamics of speech and the ensuing delta-theta band auditory activity, EEG components were filtered between 1 and 8 Hz with a third order Butterworth filter in the forward and reverse direction. Then the linear temporal response function (TRF) was estimated, a mapping between the auditory stimulus input S(t) and the evoked neural response r(t) it elicits. This linear model is formulated according to equation 2:

r_{pred} (t)=Σ_{τ}TRF (τ)S(t−τ)+ϵ(t) (2)

where ϵ(t) is the residual contribution to the evoked response not explained by the linear model and τ the amount of prestimulus samples used (0-600 ms). The prestimulus is the offset in time between the start of the auditory stimulus and the start of the neural response as measured by EEG, and is generally within the time range of 0 to 600 ms with the precise value depending on the particular subject and auditory stimulus.

Temporal response functions were estimated (step S2) by reverse correlation between stimulus and neural response timeseries (both scaled to z-units) via a boosting algorithm. This technique minimizes an error estimate ϵ(t) of the predicted response iteratively by sequential modifications to the TRF. Mean squared error was used as loss function, and after a 10-fold cross-validation procedure, the final TRF for that subject was obtained by averaging over the folds.

The temporal response function can be used to determine a signal-response latency by looking up the latency associated with a peak in the TRF as described hereinbefore (step S2). The peak search may be limited to a specified window in the TRF, for example if it is expected that the relevant peak will occur within a specific range. For example, the window may be chosen based on the age of the subject and/or the amount of distortion in the auditory stimulus, where for an increase in either or both parameters results in a window which is shifted to higher latencies. The TRF is a function for predicting at least part of the EEG response based on at least part of the signal (for example, a subset of the signal and/or a subset of the response may be chosen to be used in equation 2).

The temporal response function can be used to determine a signal-response latency difference by first determining the temporal response function for the signal in question, that is, determining a function for predicting the corresponding feature of the EEG response based on the feature of the stimulus, and then by determining the position in time of a maximum or minimum of the TRF. The position in time is then the latency difference of the feature. The search for the maximum or minimum of the TRF is preferably restricted to a specific window in time < >

### Results

### Spatial Filtering

To reduce the EEG data dimensionality while optimizing signal-to-noise ratio across subjects, a spatial filtering algorithm was implemented as described in de Cheveigné, A., & Parra, L. C., “Joint decorrelation, a versatile tool for multichannel data analysis”, NeuroImage, 98, 487-505. The resulting linear coefficients served to map the 64 channels of each subject into a single component.

### Temporal Response Function Estimation

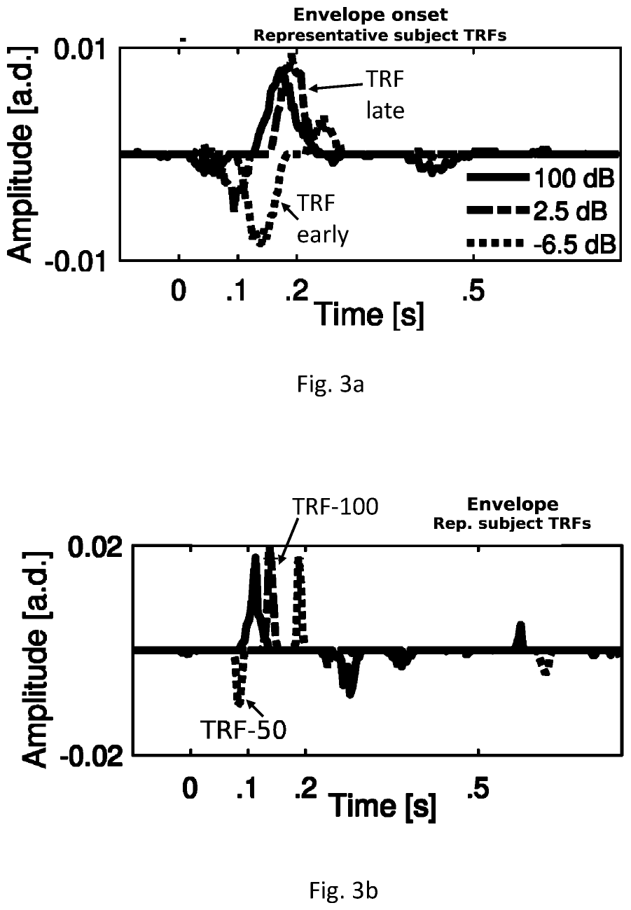

TRFs were estimated as described hereinbefore, describing the EEG response with respect to the regular or onset envelope of the speech stimulus. The TRF morphology conveys information about the timing of major processing stages of the incoming speech signal. The effect of lowering the SNR, and therefore reducing intelligibility, on the timing of peak TRF was investigated (FIG. 3a). Three TRFs are shown for different SNR levels. Using the onset envelope, the timing of the peaks show an SNR dependence, for at least early negative and late positive peaks across subjects.

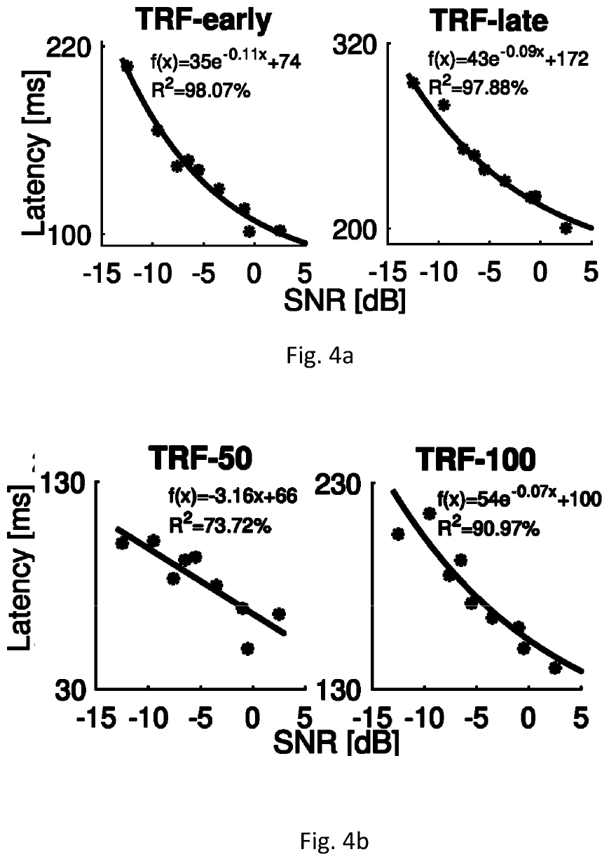

Using the onset envelope, the timing of the peaks show an SNR dependence, for at least early (around 50 ms) negative and late (around 100 ms) positive peaks (FIG. 4a) across subjects. In both cases, delays can be described by exponential growth models including peak latency information from TRFs in the noise-free limit conditions, namely +100 dB and “Story” (clean), which explains further variability (R²: TRF-early, 98.07%; TRF-late, 97.88%) of mean TRF latencies, than linear models restricted to the noise range (R²: TRF-early, 91.53%; TRF-late, 95.68%).

The findings, based on the onset envelope of the speech stimulus, were compared with the more frequently utilized regular envelope representation, for which gradual noise-induced delays have been indicated in forward models of speech encoding (Ding, N., & Simon, J. Z., “Adaptive Temporal Encoding Leads to a Background-Insensitive Cortical Representation of Speech”, Journal of Neuroscience, 33, 5728-5735). Envelope TRF peaks were generally shifted in terms of latency with respect to envelope onset TRF peaks (FIG. 3a, 3b), due to the bias inherent to half-wave rectification in onset extraction. The TRF-50 peaks suggested, as before, increasing delays with lower SNR levels (FIG. 3a, 3b). However, because grand-average reverse correlation estimates could not produce a TRF-50 peak for the noise-free limit conditions, only a linear model was tested (R²: 59.53%) in this case. The results indicated a delay rate of about 3.2 ms/dB.

With regard to the envelope-based TRF-100 peak, noise also appeared to monotonically but nonlinearly prolong peak latency, up to encoding limits where the TRF model again fails to produce relevant peak estimates at low SNRs. The progression was better described by an exponential decay model that considered latency information in the noise-free limit (R²: 90.97%), than a linear model that did not (R² 89.58%). It was confirmed that the latency of all grand-average TRF peaks increased with lower SNR, as indicated by nonzero decay constants (95% Cls: TRF-early, 0.09-0.12 dB⁻¹; TRF-late, 0.07-0.10 dB⁻¹; TRF-100, 0.04-0.09 dB⁻¹) and linear slope (95% CI: TRF-50, 1.47-4.84 ms/dB) (FIG. 3b, 4b).

Individual noise-induced delay measures estimation

For most conditions, individual TRFs exhibited at least one positive peak in the 0-400 ms window. Across noise levels, TRFs were inspected on a per subject basis in this window, in order to identify the TRF-late positive peak (FIG. 3a). For a subset of 12 subjects, the earlier peak of opposite polarity (TRF-early), was also identified in consistent manner across conditions, and also submitted to further analysis. Latencies of the TRF-late (and where applicable, TRF-early) peaks were estimated per SNR level by a cross-correlation procedure with respect to the respective peak in the grand average model. Peak latency progressions by noise were found to be adequately described by exponential fit models at the single subject level (FIGS. 4a, 4b), R² measures in the 95.4±4.6% range for TRF-early latency data (N=12), and 85.5±18.6% for TRF-late peaks (N=28). This suggests consistent noise-related delay for both peaks across individuals, as indicated by nonzero growth constants at the subject level (95% CI: TRF-early, 0.079-0.143 dB⁻¹; TRF-late, 0.085-0.139 dB⁻¹).

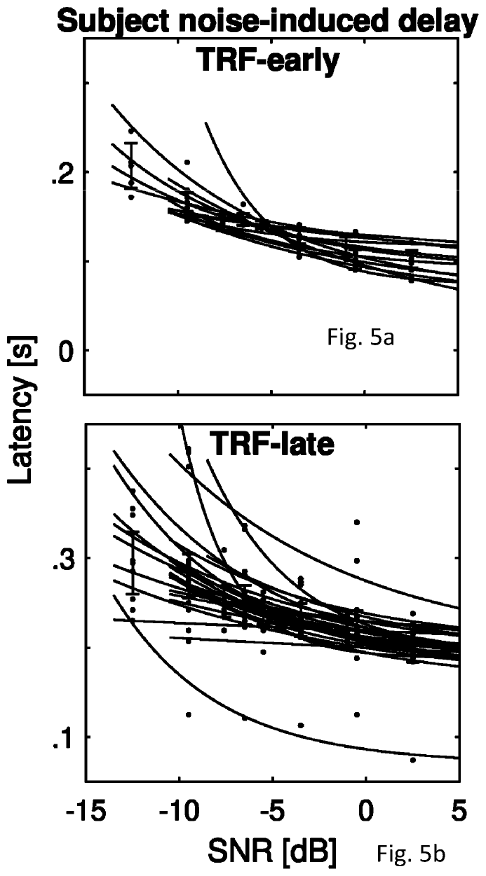

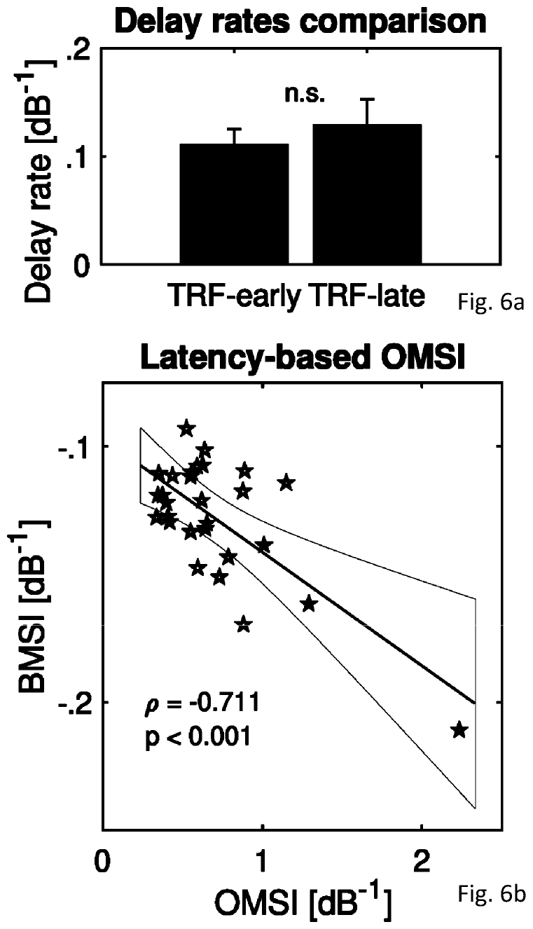

A Wilcoxon rank-sum test was done to analyse whether, when both are present, TRF-early versus TRF-late peaks are delayed at different rates over increasing noise. The hypothesis that the peaks have different latency delay rates as a consequence of noise was not rejected (p=0.67; N=12) (FIG. 5a). Overall the results suggest that out of the examined peaks, the EEG TRF-late peak, may provide the most robust basis to inspect noise-induced delays per subject. Given that no differential contribution from earlier processes was expected, this component alone was then further inspected for the purposes of estimating an objective measure of speech intelligibility.

Latency-based objective measure of speech intelligibility

Noise-induced delay estimates can be measured in absolute terms as the difference between the latency for a feature of a first stimulus with a given noise level and the latency for the feature in a reference stimulus. In the case of inter-subject variable latencies at any given condition (FIG. 4b), including the reference auditory stimulus, these may also be more adequately expressed in terms of change ratios.

The change ratios were computed by referencing each noise condition with respect to the average TRF peak latencies in the Story and the SNR-100 dB conditions (step S3), which together provided a relatively more robust representation of Matrix speech processing in the noise-free limit. In this example the latencies are compared by the change ratio, or division function, but as described elsewhere herein the present invention is not limited thereto and other functions are possible for comparing the latencies.

To obtain an objective measure of speech intelligibility, the relationship between the SNR level and the noise-induced latency change ratios (equivalently expressed in dB units) was inspected per subject. Across subjects, change ratios cr(SNR) were better described by an exponential model according to equation 3:

cr(SNR)=a^{-b×SNR} (3)

with the noise-free limit referenced to zero (R²=0.8715) than a linear model (R²=0.8072). For subject i single-subject parameters aᵢ and bᵢ were estimated, corresponding to the subject's delay rate at 0 dB versus clean and the delay growth rate, respectively. It was hypothesized that individual differences in the increase in latency with SNR was a possible contributor to the subject's ability to cope with noise and therefore can be a proxy of the SRT.

It was found that aᵢ parameters, particularly their reciprocals, could be used to predict behaviorally-obtained speech reception thresholds (SRT) in subjects (step S4). The correlation between SRTᵢ and aᵢ reciprocals was significant (Pearson's r=−0.711, p=2.2×10⁻⁵), indicating that the individuals' rate of change when equal power noise is removed from the speech signal may predict their tolerance to noise or, in other words, the speech intelligibility. Subjects whose processing was delayed the most were also the least tolerant to noise.

By fitting a function to a dataset of aᵢ values and corresponding behaviourally measured SRT values, this function can be used to predict the SRT of a subject based on their aᵢ value. Thus the SRT of a subject can be determined objectively, that is, without needing to carry out a behavioural measure of the SRT of the subject.

The measure of speech intelligibility may be a relative measure of speech intelligibility. For example, the method may comprise providing a second time-dependent signal based on a second auditory stimulus having a second noise parameter value which is different to the first noise parameter value and a corresponding second measured EEG response, determining a temporal response function for predicting at least part of the second EEG response based on the second signal, and performing a cross-correlation of the first TRF and the second TRF in order to determine the signal-response latency difference as a relative signal-response latency difference. The relative signal-response latency difference is the latency of the maximum (or the minimum) of the cross-correlation. The relative signal-response latency difference can then be used to determine a relative speech intelligibility using methods as described hereinbefore with respect to determining a speech intelligibility measure.

Referring again to FIG. 1a, embodiments of the present invention provide an apparatus for carrying out a method as described hereinbefore, the apparatus comprising at least a control module for receiving and processing an auditory stimulus and an EEG response. The control module comprises a processor comprising instructions for processing the auditory stimulus and EEG response and optionally a memory for storing auditory stimuli and responses. The processor may retrieve the stimulus and response from the memory before processing. The control module may comprise input means for receiving the auditory stimulus and response from external sources such as measurement equipment and/or databases stored elsewhere.

According to embodiments of the present invention there is provided a computer program comprising instructions which, when the program is executed by a computer, cause the computer to carry out a method as described herein. The present invention also encompasses a computer-readable medium comprising instructions which, when executed by a computer, cause the computer to carry out a method as described herein.

For clinical uses, it is essential to reliably measure speech intelligibility. By using EEG methods the subjects' ability to parse speech in noise is directly addressed. The methods described herein allow the use of time domain information which can complement existing EEG measures of cortical tracking of speech as objective measures of speech intelligibility for audiology purposes. In cortical tracking methods, such measures typically indicate a lower bound on the degree to which speech features may be represented in cortical activity. Their interpretation is therefore tied to the stimulus as much as it is to a general property of the auditory system. The methods described herein comprise a means to extract precise temporal estimations of how the auditory system processes speech in general, by taking advantage of cortical locking to acoustic edge information in combination with sparse response function estimation methods. This results in a simple and versatile tool to measure critical timing information as a property of the system itself.

The methods described herein enable extraction of high temporal resolution information consistently across listeners, by modeling noise-induced delays on speech processing. When tested against a behavioral test of speech intelligibility using a validated corpus of spoken sentences, it is found that behavioural intelligibility tests correspond to noise-induced delay trendline estimates. In particular, the ratio of processing delays between noise-free and equalized (i.e. 0 dB) noise conditions was found to correlate the speech reception threshold. The latter indicates the stimulus SNR at which the subject has understood 50% of the words, and is considered the current gold standard in speech audiology for both research and clinical purposes.

1. A method of estimating speech intelligibility comprising:
providing at least a first time-dependent signal derived from a first auditory stimulus wherein the first auditory stimulus has a first noise rating and a corresponding first measured EEG response;
comparing at least part of the first signal with at least part of the first measured EEG response in order to determine a first signal-response latency difference;
providing a second signal derived from a second auditory stimulus, wherein the second auditory stimulus has a second noise rating which is different to the first noise rating, and a corresponding second measured EEG response; comparing at least part of the second signal with at least part of the second measured EEG response in order to determine a second signal-response latency difference;
comparing the first signal-response latency difference to the second signal- response latency difference; and
deriving a measure of speech intelligibility based on the comparison of the first signal-response latency difference and the second signal-response latency difference.

2. A method according to claim 1, wherein the step of comparing at least part of the first signal and at least part of the EEG response comprises determining a temporal response function for predicting at least part of the EEG response based on the first signal.

3. A method according to claim 1, wherein the step of comparing at least part of the first signal and at least part of the EEG response comprises applying a plurality of test latency differences to the first signal and determining a quantitative measure of the similarity of at least part of the first signal and at least part of the EEG response for each applied test latency difference, and wherein the signal-response latency difference is determined as the latency difference at which the quantitative similarity measure is greatest.

4. A method according to claim 2, wherein the first auditory stimulus has a first noise rating, and wherein the method further comprises:
providing a second time-dependent signal based on a second auditory stimulus having a second noise rating which is different to the first noise rating and a corresponding second measured EEG response;
comparing at least part of the second time-dependent signal and at least part of the second EEG response, wherein the comparison comprises determining a temporal response function for predicting at least part of the second EEG response based on the second signal; and
performing a cross-correlation of the first temporal response function and the second temporal response function in order to determine the signal-response latency difference as a relative signal-response latency difference, wherein the relative signal-response latency difference is the latency of the maximum or minimum of the cross-correlation.

5. A method according to claim 1, wherein the second auditory stimulus is noise-free.

6. A method according to claim 1, further comprising providing a third time-dependent signal derived from a third auditory stimulus, wherein the third auditory stimulus has a third noise rating which is different to the first noise rating and the second noise rating, and a corresponding third measured EEG response;
comparing at least part of the third signal and at least part of the third EEG response in order to determine a third signal-response latency difference; comparing the third signal-response latency difference to the first and/or second signal-response latency difference; and
deriving a measure of speech intelligibility based on the comparison of the third signal-response latency difference and the first and/or second signal- response latency difference.

7. A method according to claim 1, wherein the noise rating is the signal-to-noise ratio.

8. A method according to claim 1, wherein comparing the signal-response latency differences comprises supplying the signal-response latency differences as inputs to a comparison function which comparison function produces a single output.

9. A method according to claim 8, comprising the step of obtaining at least at least two sets, a set for the first auditory stimulus and a set for the second auditory stimulus, each set comprising a comparison function output and a corresponding noise rating for the respective stimulus.

10. A method according to claim 8, wherein determining a measure of speech intelligibility comprises fitting a function to the comparison function output-noise rating data and determining the speech intelligibility based on a parameter of the fitted function.

11. A method according to claim 8, wherein the comparison function computes the first signal-response latency difference divided by the first signal-response latency difference or vice versa, or the difference between the signal-response latency differences.

12. A method according to claim 10, wherein the fitted function is a linear or an exponential or a sigmoid function.

13. A method according to claim 1, wherein the signal is the envelope of the stimulus or the derivative of the envelope of the stimulus.

14. A computer program comprising instructions which, when the program is executed by a computer, cause the computer to carry out the method of claim 1.

15. An apparatus configured to carry out a method according to claim 1, the apparatus comprising at least a control module for receiving and processing an auditory stimulus and an EEG response.
